(19) United States
(12) Patent Application Publication
(10) Pub. No.: US 2010/0121463 A1
(43) Pub. Date: May 13, 2010

(54) BIOABSORBABLE AND BIOACTIVE COMPOSITE MATERIAL AND A METHOD FOR MANUFACTURING THE COMPOSITE

(75) Inventors: Pertti Törmälä, Tampere (FI); Mikko Huttunen, Tampere (FI); Nureddin Ashammakhi, Helsinki (FI); Mikko Tukiainen, Nokia (FI); Heimo Ylänen, Turku (FI); Mikko Hupa, Turku (FI); Minna Kellomäki, Kangasala (FI)

Correspondence Address: VENABLE LLP, P.O. BOX 34385, WASHINGTON, DC, 20043-9998, US

(73) Assignee: BIORETEC OY, Tampere (FI)

(21) Appl. No.: 11/919,492

(22) PCT Filed: Apr. 27, 2006
(86) PCT No.: PCT/FI2006/050171

§ 371 Date: Jan. 28, 2010

(30) Foreign Application Priority Data
Apr. 27, 2005 (FI) 20055194

(51) Int. Cl. A61F 2/02

(52) U.S. Cl. 623/23.75

(57) ABSTRACT

The present invention relates to a bioabsorbable and bioactive composite material for surgical musculoskeletal applications comprising a bioabsorbable polymeric matrix material which is reinforced with bioabsorbable polymeric fibers and bioabsorbable ceramic fibers. The surgical bioabsorbable polymeric matrix material is reinforced with the bioabsorbable polymeric fibers and the bioabsorbable ceramic fibers from which at least a portion is longer than 150 μm. The invention also relates to a method for manufacturing a bioabsorbable and bioactive composite material.

### FIELD OF THE INVENTION

The present invention relates to a bioabsorbable and bioactive composite material for surgical musculoskeletal applications comprising a polymeric matrix material which is reinforced with bioabsorbable polymeric fibers and bioabsorbable ceramic fibers.

### BACKGROUND OF THE INVENTION

Biostable or bioabsorbable devices are used in surgery for musculoskeletal applications, such as e.g. (a) screws, plates, pins, tacks or nails for the fixation of bone fractures and/or osteotomies to immobilize the bone fragments for healing, (b) suture anchors, tacks, screws, bolts, nails, clamps and other devices for soft tissue-to-bone (or- into-bone) and soft tissue-to-soft tissue fixation or (c) cervical wedges and lumbar cages and plates and screws for vertebral fusion and other operations in spinal surgery.

Most biostable devices are typically made of metallic alloys (see e.g. M. E. Müller, M. Allgöwer, R. Schneider, H. Willenegger “Manual of Internal Fixation”, Springer-Verlag, Berlin Heidelberg New York 1979). However, there are several disadvantages in the use of metallic implants. One such disadvantage is bone resorption caused by high modulus bone plates and screws, which carry most of the external loads, leading to stress protection produced by the modulus mismatch between metals and bone. Another disadvantage is the possibility of corrosion. Therefore, it is recommended that surgeons should remove metallic devices (like bone plates and screws) in a second operation once the fracture has healed.

Bioabsorbable polymeric fracture fixation devices have been developed and studied as replacements for metallic implants (see e.g. S. Vainionpää, P. Rokkanen, P. Törmälä, “Surgical Applications of Biodegradable Polymers in Human Tissue”, Progress in Polymer Science, Vol. 14, 1989, pp. 679-716).

The advantages of these devices are that materials are resorbed in the body and the degradation products exit via metabolic routes. Hence, a second operation is not required. Additionally, the strength and the stiffness (modulus) of the bioabsorbable polymeric devices decreases when the device degrades and hence the bone is progressively loaded more and more, which promotes bone regeneration (according to Wolff's law).

One limitation of the prior art bioabsorbable materials and devices is their relatively low modulus and strength. In the case of cortical bone fractures, for example, non-reinforced poly lactic acid (PLLA) plates and screws are initially too weak to permit patient mobilization (see e.g. J. Eitenmüller, K. L. Gerlach, T. Schmickal, H. Krause, “An in Vivo Evaluation of a New High Molecular Weight Polylactide Osteosynthesis Device”, European Congress on Biomaterials, Bologna Italy, September 14-17, 1986, p. 94).

Törmälä et al. have developed self-reinforced bioresorbable polymeric composites to improve the strength of bioresorbable polymer devices. These show relatively good mechanical properties: e.g. bending strength of 360±70 MPa and bending modulus of 12±2 GPa, respectively, have been reported (see P. Törmälä, “Biodegradable Self-Reinforced Composite Materials; Manufacturing, Structure and Mechanical Properties”, Clinical Materials, Vol. 10, 1992, pp. 29-34). However, the reported modulus values are still below the modulus values of strong cortical bone (see e.g. S. M. Snyder and E. Schneider, “Estimation of Mechanical Properties of Cortical Bone by Computed Tomography”, Journal of Orthopedic Research, Vol. 9, 1991, pp. 422-431, giving the bending modulus of 17.5 GPa for human tibial bone). It is desirable that the modulus of a fixation device is at least as high as the modulus of cortical bone so that the fixation system is practically isoelastic with the bone, which gives the possibility to natural, controlled micromotions of fixed bone fragments in relation to each other. Such natural micromotions accelerate the fracture consolidation and ossification (healing) and reduce the risks of too big micromotions (leading to fibrous non-union) or too small micromotions (leading to stress-protection atrophy and increased porosity of healing bone).

A common property of most polymeric implants is the lack of bony ongrowth to the materials. In contrast, such bone apposition is produced by bioactive ceramics, such as bioactive glasses (see e.g. O. H. Andersson, K. H. Karisson, “Bioactive Glass, Biomaterials Today and Tomorrow”, Proceedings of the Finnish Dental Society Days of Research, Tampere, Finland, 10-11 November 1995, Gillot Oy, Turku, 1996, pp. 15-16). By adding (compounding) bioactive ceramics, such as bioactive glasses to polymers to produce composites, the bioactivity of the material can be improved. This effect has been demonstrated in dental composites and bone cement (see e.g. J. C. Behiri, M. Braden, S. N. Khorashani, D. Wiwattanadate, W. Bonfield, “Advanced Bone Cement for Long Term Orthopaedic Applications”, Bioceramics; Vol. 4, ed. W. Bonfield, G. W. Hastings and K. E. Tanner, Butterworth-Heinemann Ltd., Oxford, 1991, pp. 301-307).

Zimmerman et al. developed unidirectional composites of poly-L-lactide matrix reinforced with calcium/phosphorous oxide (CaP) based biodegradable glass fibers. This composite showed good initial strength and modulus values, e.g. tensile strength 200.3±7.1 MPa, tensile modulus 29.9±2.2 GPa, bending strength 161.3±8.8 MPa, bending modulus 27.0±0.3 GPa. However, the strength reinforcing effect of CaP fibers in hydrolytic conditions (in vitro: a tris-buffered saline of pH 7.4 at 37° C.) was lost totally after 23 days of immersion, while only 35% of the initial strength and 45% of the initial modulus was retained. It can be concluded that this composite, which was reinforced with long ceramic fibers, lost its strength too rapidly to be applied as a raw material of bone fracture fixation devices. See M. Zimmerman, T. Guastavin, J. R. Parsons, H. Alexander and T. C. Lin: “The in vivo biocompatibility and in vitro degradation of absorbable glass fiber reinforced composites”, 12^{th} Ann. Meeting Soc. Biomater., p. 16, Minneapolis-St. Paul, Minn., USA (1986).

A. Saikku-Bäckström et al. Studied in vivo and in vitro hydrolysis of poly-96U4D-lactide fiber reinforced poly-96L/4D-lactide matrix (fibrillated SR-PLA 96) rods (diam. 1.1 mm). The rods had an initial bending strength of 225 MPa and a bending modulus of 8.4 GPa. After 168 days (24 wk) of in vitro hydrolysis in buffered saline at 37° C., the bending strength was still 86.7% (195 MPa) of the initial value. The bending modulus of same rods after 168 days hydrolysis in the above conditions was still 82.1% (6.9 GPa) of the initial value. It can be concluded that these bioabsorbable polylactide fiber reinforced polylactide rods had a good bending strength and a bending modulus retention in hydrolytic conditions, even if the initial bending modulus was stilt far below the bending modulus of cortical bone. See: A. Saikku-Bäckström et al. in J. Mater. Sci: Mater. Med. 10 (1999) p. 1-8.

Bioabsorbable composites of hydroxyapatite and copolymers of polyhydroxybutyrate and polyhydroxyvalerate have been described by C. Doyle, K. E. Tanner, W. Bonfield, see “In Vitro and in Vivo Evaluation of Polyhydroxybutyrate and of Polyhydroxyvalerate Reinforced with Hydroxyapatite”, Biomaterials, Vol. 12, 1991, pp. 841-847). The main limitation of these bioabsorbable composites is their inadequate mechanical strength for large bone fracture fixation. Also, the use of hydroxyapatite and polylactic acid composites has been reported. See Y. Ikada, H. H. Suong, Y. Shimizu, S. Watanabe, T. Nakamura, M. Suzuki, A. T. Shimamoto, “Osteosynthetic Pin”, U.S. Pat. No. 4,898,186, 1990. Using existing elements the composites still have quite moderate mechanical strength and modulus.

Prior art also teaches biodegradable and bioactive composites with at least one resorbable polymeric reinforcing element and at least one ceramic reinforcing element with a particle size between 2 μm and 150 μm (see P. Törmälä, M. Kellomäki, W. Bonfield, K. E. Tanner, “Bioactive and

Biodegradable Composites of Polymers and Ceramics or Glasses and Method to Manufacture such Composites”, EP 1 009 448 B1). Even if these composites show improved strength and modulus in comparison to many non-reinforced bioactive polymer—ceramic composites, their modulus values (8.3 GPa-14.2 GPA) are still clearly lower than the modulus values of strong cortical bone (see e.g. Snyder and Schneider above).

Q.-Q. Qin et al. describe in WO 2004049904 a flexible, bioactive mesh comprising glass fibers and first resorbable polymer fibers in which said glass fibers are interwoven with said first resorbable polymer fibers. However, this is a low modulus material because there is no matrix polymer which could transfer loads from fibers to each other and could prevent fibers from moving in relation to each other when external forces are directed to the mesh.

Accordingly, prior art teaches that (a) bioabsorbable composites, reinforced with absorbable glass fibers, have a high initial bending modulus, but they rapidly lose their strength and modulus in vitro, and (b) bioabsorbable composites reinforced with bioabsorbable polymer fibers have a good strength retention in vitro, but their initial bending modulus values are well below the modulus values of cortical bone, and (c) bioabsorbable composites reinforced with bioabsorbable polymer fibers and with ceramic reinforcing elements with a particle size between 2 μm and 150 μm, also have initial bending modulus values below the modulus values of cortical bone.

Accordingly, there exists a need for strong bioabsorbable, composite materials with high initial bending modulus and bending strength to guarantee the safe initial consolidation and healing of bone fractures. There exists further a need for such materials which additionally retain the high strength values under hydrolytic conditions at 37° C. over four weeks to guarantee the safe consolidation and healing of bone fractures. There exist further a need for such materials, which additionally are osteoconductive, which means that they promote and facilitate bone healing.

Such materials with high initial modulus and good strength retention in vitro are useful in manufacturing of e.g. bone fracture fixation devices, because high initial modulus and strength retention under hydrolytic conditions provide the devices with an initial isoelastic behaviour in comparison to the healing bone, which means stronger control of micromotions in the healing bone, leading to an improved healing and to a lower risk of non-unions during healing. The high strength of the implant guarantees safe progress of healing after the early consolidation of the fracture.

### SUMMARY OF THE INVENTION

Now, we have surprisingly found that bioabsorbable, bioactive composites with the high initial modulus and strength (specially high impact strength) and good strength retention behaviour in vitro under hydrolytic conditions are obtained by reinforcing a bioabsorbable polymer matrix both with bioabsorbable polymeric fibers and with bioabsorbable ceramic fibers, of which at least a portion is longer than 150 μm.

We shall describe composite materials and devices of the invention, which comprise at least one polymeric matrix phase, at least one bioactive ceramic reinforcing long fiber phase and at least one bioabsorbable polymeric reinforcing long fiber phase. The reinforced composite materials and devices described in this invention have an improved combination of mechanical strength and modulus properties when compared to reinforced and non-reinforced materials and devices of prior art, because reinforcement with long ceramic and polymeric fibers will increase both the modulus and strength retention of the material when compared to prior art materials. Thanks to the controlled manufacturing stages of combining of matrix and ceramic reinforcing fibers as well as polymeric reinforcing fibers, the amount of both reinforcing fiber types can be easily controlled. This is an advantage, because the ratio of the elements will affect the mechanical properties of the device. Also, the amount of the ceramic reinforcing fibers will affect the bioactivity of the device.

Bioabsorbable polymeric long fibers and ceramic fibers differ significantly from each other in their mechanical behaviour.

Polymeric long fibers are tough and strong, and therefore they can increase the toughness and strength values, such as the tensile, bending and impact strength of composites.

Ceramic long fibers have high stiffness and therefore they can increase the stiffness (modulus values) of even polymer fiber reinforced composites.

By combining long bioabsorbable polymeric fibers and long ceramic fibers in different ways with the bioabsorbable polymer matrix, we can obtain bioabsorbable high modulus composites with different property combinations.

Reinforcing of the bioabsorbable polymer matrix both with bioabsorbable, polymeric long fibers and with bioabsorbable ceramic long fibers will provide the materials with unique properties: for example, when a fixation implant (e.g. a pin or a screw) for a bone fracture is made of this material, the implant has first a high strength and modulus when both polymeric and ceramic fibers reinforce the implant. This means that the fixation implant is secure and gives an optimal protection for the early consolidation of the bone fracture. Thereafter, typically after some weeks, the ceramic fibers will lose their reinforcing effect, so that only bioabsorbable fibers reinforce the matrix. As a consequence, the strength and the modulus of the implants decrease progressively. However, this decreasing is not as drastic as in prior art materials, since bioabsorbable reinforcing fibers still maintain the strength and ductility of the implant, typically up to 2-6 months after the implantation. This secures the final healing of bone fractures for which the ceramic fibers gave the early strong protection for early consolidation.

Besides the long polymeric fibers and long ceramic fibers, the composite of the invention may include bioabsorbable ceramic fibers having a length which is 150 μm or shorter, or bioabsorbable ceramic powder.

In addition to the above-mentioned findings, we shall describe laminates which are reinforced with the ceramic fibers and the polymeric fibers.

### BRIEF DESCRIPTION OF THE DRAWINGS

FIGS. 1a, 1b and 5 show schematic perspective views of typical composite materials of the invention,

FIGS. 2 and 3 show schematic cross-sectional views of composite materials of the invention,

FIG. 4 shows a perspective view of the cage-like embodiment of the invention,

FIG. 6 shows schematically a formation of a laminate material according to an embodiment of the present invention,

FIG. 7 shows the 3-point bending strength and the bending modulus as a function of the amount of the bioabsorbable glass fibers,

FIGS. 8-10 show structures of laminate materials in a perspective view, and

FIG. 11 shows results of IZOD impact strength measurements.

### DESCRIPTION OF THE PREFERRED EMBODIMENTS

The present invention relates to bioabsorbable materials and devices for musculoskeletal applications, such as e.g. bone fracture or osteotomy fixation, soft tissue (like tendon)-to-bone fixation, soft tissue-to-soft tissue fixation and guided bone regeneration applications, such as vertebral fusion. Unlike other materials used in prior art, the composites of this invention have two different reinforcing, bioabsorbable, long fiber phases and at least one matrix phase. One reinforcing long fiber phase is referred to as the polymeric reinforcing fiber phase and the other as the ceramic reinforcing fiber phase. The matrix component can be any bioabsorbable or bioerodible polymer, copolymer or polymer alloy (mixture of two or more polymers or copolymers). The polymer can be synthetic or “semisynthetic”, which means polymers made by chemical modification of natural polymers (such as starch). Typical examples of polymers, which can be used in this invention, are listed in Table 1 below.

The polymeric reinforcing fibers and ceramic reinforcing fibers are recognizable and distinguishable in the final product.

The diameter of the reinforcing polymeric long fibers can vary typically between 4 μm and 800 μm, preferably between 20 μm and 500 μm. The most useful range is between 30 μm and 70 μm. Useful polymers for the polymeric reinforcing fibers include several of those listed in Table 1.

The ceramic reinforcing fibers typically comprise biodegradable, bioactive long fibers of bioactive glass with diameters typically from 1 μm to 800 μm and preferably from 5 μm to 500 μm. The diameters of ceramic reinforcing fibers are often in the range between 1 μm and 20 μm. The fibers with a diameter less than 10 μm, are of importance. Typical examples are listed in Table 2. They can be used as long single fibers, as yarns, braids, bands or as different types of fabrics made by the methods of textile technology.

Ceramic fibers and/or polymeric fibers may also be introduced in the polymer or composite structure in the form of prefabricated products, such as prepregs, etc., manufactured by means of techniques of the polymer composite technology, in addition to the methods of textile technology.

The fibers of this invention are long, which means that their length is many times (10× or more) their diameter. They are typically longer than 150 μm, preferably longer than 2 millimeters and more preferably longer than 30 millimeters. At their best, the fibers are continuous so that their length is the same (or greater) than the longest dimension of the device (the fibers can be longer than the longest dimension of the device if the fibers are e.g. twisted, wound or braided).

The ceramic reinforcing fibers also act as a bioactive, bony ongrowth agent and provide a reservoir of calcium and phosphate ions, thus accelerating the bone healing. These ions may also have a buffering effect on the acidic degradation products of the resorbable polymeric components of the composite. While the matrix polymer degrades, bone can attach to the residual ceramic or glass material. The amount of polymeric reinforcing fibers or ceramic reinforcing fibers in the composite is from 10wt % to 90 wt %, preferably from 20wt % to 70 wt %.

The materials of the invention may contain various additives and modifiers which improve the performance of the composite or its processability. Such additives include surface modifiers to improve the attachment between the polymeric and ceramic components. The devices may also contain pharmaceutically active agents, such as antibiotics, chemotherapeutic agents, wound-healing agents, growth hormones and anticoagulants (such as heparin). These agents are used to enhance the bioactive feature of the composite, to make it multifunctional and to improve the healing process of the operated tissues.

Manufacturing of the composite can be performed by any suitable processing methods of plastics technology, polymer composite technology and/or textile technology. The matrix polymer and the polymeric reinforcing fibers and the ceramic reinforcing fibers (bioceramic or bioactive glass) can be mixed together by mechanical mixing, melt mixing or solvent mixing. The polymeric and/or ceramic reinforcing fibers can be used as plain fibers or in a modified form: for example, as braided, knitted or woven to two- or three-dimensional structures (together or as separate fabrics) or in the form of preforms such as prepregs: The mixture of matrix and the polymeric reinforcing fibers and the ceramic reinforcing fibers can be combined by mixing, by coating or by using a solvent as an intermediate to preform the material (prepreg). The material preform or final device can be produced by various techniques including compression molding, transfer molding, filament winding, pultrusion, melt extrusion, mechanical machining or injection molding to any desired shape.

When the polymeric and/or ceramic long reinforcement fibers of the composites of the invention are continuous the composites have better mechanical properties than short or non-continuous long fibre reinforced bioabsorbable composites. One of the most important factors is thus the absence of fiber ends in the continuous fiber reinforced composites, which are sites for crack iniatiation during fracture due to mechanical loading.

Processing methods include e.g.:
postpregging methods
prepregging methods:
   film stacking
   incapsulated powder impregnation
   melt impregnation
   powder impregnation
   co-weaving
   comingling

The post- and prepregs are placed in controlled orientation during the manufacture of the composite. Next, pressure and heat are applied, resulting in the total or partial melting of the bioabsorbable matrix and the forming of the composite structure after cooling. Continuous bioabsorbable polymer and ceramic fiber reinforced composites can be produced e.g. by compression molding, thermoforming, filament winding, tape laying, braiding and pultrusion methods and by several combinations of these methods. Such methods are disclosed e.g. in the publication (Doctoral Thesis): E. Suokas, “Processing, microstructure and properties of thermotropic liquid crystalline polymers and their carbon fibre composites”, Tampere University of Technology, Publication 267, Tampere, Finland 1999, 269 pp.

Due to controlled manufacturing stages of mixing and combining of the matrix and the ceramic reinforcing fibers as well as combining the polymeric reinforcing fibers, the amount of both reinforcing fiber types can be easily controlled. This is an important advantage, because the ratio of the polymeric and ceramic fibers affects the mechanical strength and modulus properties of the device. Also, the amount of the ceramic reinforcing fibers affects the bioactivity of the device. There should be a sufficient amount of bioceramic or bioactive glass fibers to yield bony on—and ingrowth.

Fiber reinforced composite devices described in this invention have improved mechanical properties compared to non-reinforced devices, because reinforcement will change the behavior of the materials from brittle to ductile and thus make the reinforced device more reliable under loading. This feature is very important for load bearing applications, such as bone fracture fixation devices. For example, non-reinforced polylactic acid devices typically have three-point bending strengths of 35-40 MPa and modulus of 3.5-4.0 GPa, and particulate reinforced (hydroxyapatite) polylactic acid devices have values of 25-30 MPa and 5.0 GPa, respectively. When the composite is made of three components: polymer matrix, reinforcing polymer fibers and ceramic particulate filler, the modulus can be increased up to 8-10 GPa (M. Kellomäki et al., 13^{th} Eur. Conf. Biom., Abstracts, Gothenburg, Sweden, Sept. 4-7, 1997, p. 90). However, using long polymer fiber reinforcement and long ceramic fiber reinforcement, under the present invention, the strength and modulus values of composites can still be increased to a significant extent.

One useful bioabsorbable and bioactive composite is a laminate comprising at least two layers. The composite may comprise
polymeric layers which are not reinforced,
polymeric layers comprising reinforcing fibers, or
layers of ceramic fibers.

The polymeric layers, which are not reinforced, may comprise for example poly-L/DL-lactide 70/30. The polymeric layers, which comprise reinforcing fibers, may also comprise for example poly-L/DL-lactide 70/30. One polymeric layer may comprise both ceramic and polymeric fibers, or only ceramic or polymeric fibers. The reinforcing fibers are usually continuous but they can be staple fibers as well. It is also possible that the staple fibers are spun to a yarn which is used for reinforcing in a continuous form.

The fiber orientation in the polymeric layer can vary. The reinforcing fibers can be parallel or traverse to the longitudinal axis of the polymeric layer or they may form an angle with the longitudinal axis. A random orientation is also possible.

The layers of ceramic fibers, such as bioactive glass fibers, may be formed of parallel fibers which are adhesively attached to each other.

Besides the above-mentioned variations, the reinforcing fibers may form textile structures, such as braidings or woven fabrics.

The fiber orientation in the superimposed layers may differ from each other. In such a manner structures, which are strong to all directions, will be produced. Thus the structures resist very well torsional forces.

The layers of the laminate are laminated together by using heat and pressure. The number of the layers to be laminated together varies depending on the desired end use. Those laminates are useful for example in surgical fixation devices, such as fixation plates for bone fractures, or in implants for ossifying vertebrae.

TABLE 1
Bioabsorbable, (resorbable) polymers, copolymers and terpolymers
suitable for composites of the invention (Useful as materials for
the bioabsorbable polymeric fibers and for the bioabsorbable
polymeric matrix).

```
    Polyglycolide (PGA)
    Copolymers of glycolide:
Glycolide/L-lactide copolymers(PGA/PLLA)
Glycolide/trimethylene carbonate copolymers (PGA/TMC)
    Polylactides (PLA)
    Stereocopolymers of PLA:
Poly-L-lactide (PLLA)
Poly-DL-lactide (PDLLA)
L-lactide/DL-lactide copolymers
    Other copolymers of PLA:
Lactide/tetramethylglycolide copolymers
Lactide/trimethylene carbonate copolymers
Lactide/d-valerolactone copolymers
Lactide/ε-caprolactone copolymers
    Terpolymers of PLA:
Lactide/glycolide/trimethylene carbonate terpolymers
Lactide/glycolide/ε-caprolactone terpolymers
PLA/polyethylene oxide copolymers
    Polydepsipeptides
    Unsymmetrically 3,6-substituted poly-1,4-dioxane-2,5-diones
    Polyhydroxyalkanoates:
Polyhydroxybutyrates (PHB)
PHB/b-hydroxyvalerate copolymers (PHB/PHV)
    Poly-b-hydroxypropionate (PHPA)
    Poly-p-dioxanone (PDS)
    Poly-d-valerolactone
    Poly-e-caprolactone
    Methylmethacrylate-N-vinyl pyrrolidone copolymers
    Polyesteramides
    Polyesters of oxalic acid
    Polydihydropyrans
    Polyalkyl-2-cyanoacrylates
    Polyurethanes (PU)
    Polyvinylalcohol (PVA)
    Polypeptides
    Poly-b-malic acid (PM LA)
    Poly-b-alkanoic acids
    Polycarbonates
    Polyorthoesters
    Polyphosphates
    Polyanhydrides
```

TABLE 2
Bioceramics and glasses suitable for composites of the invention.

```
    Hydroxyapatite
    Calcium phosphates:
Tricalcium phosphates
    Bioactive glasses
    Bioactive glass-ceramics
```

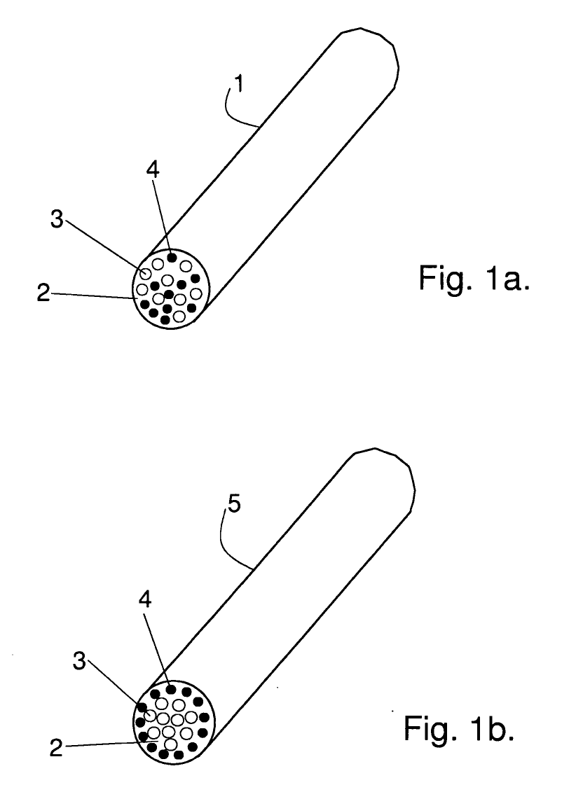

FIG. 1a shows a cylindrical bar 1 comprising a polymer matrix 2, long polymer fibers 3 and slightly thinner ceramic fibers 4 bound by the polymer matrix 2.

FIG. 1b shows a high bending modulus cylindrical bar 5 with polymer fibers 3 in the core of the bar and ceramic fibers 4 in the surface area of the bar.

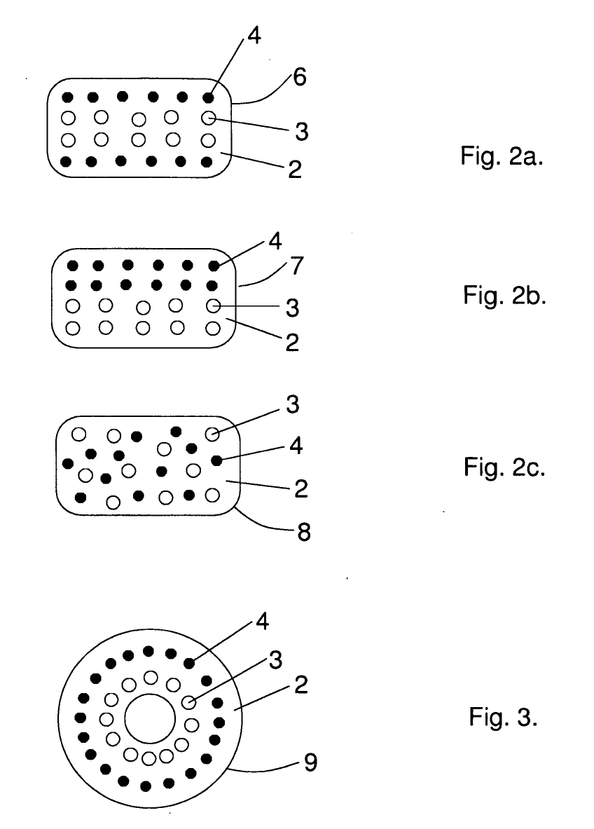

FIG. 2 illustrates, as an example, cross-sections of rectangular bars with different arrangements of long bioabsorbable polymeric and ceramic reinforcement fibers. FIG. 2a shows the cross-section of a bar 6 with a polymer matrix 2, in which polymeric fibers 3 are found in the inner area of the bar 6 and ceramic fibers 4 near the surfaces of the bar 6.

FIG. 2b shows a cross-section of a bar 7 with the matrix 2, in which polymeric fibers 3 are found near the lower surface of the bar 7 and ceramic fibers 4 near the upper surface of the bar 7.

FIG. 2c shows a cross-section of a bar 8 with the matrix 2, in which polymer fibers 3 and ceramic fibers 4 are distributed randomly into the matrix 2 of the bar.

FIG. 3 illustrates a cross-section of a tubular implant 9 with a parallel, continuous fiber reinforcement by polymeric fibers 3 and ceramic fibers 4, both embedded in polymer matrix 2, according to the invention.

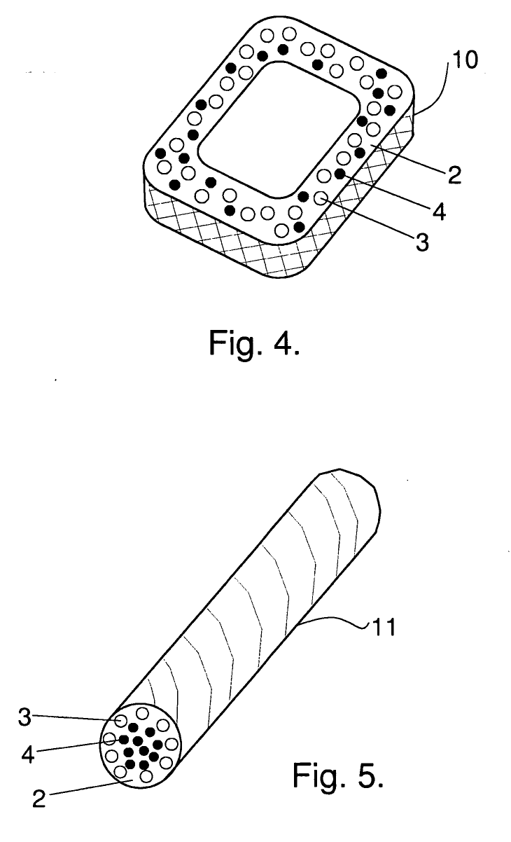

FIG. 4 illustrates an advantageous embodiment of the invention. Here a bioabsorbable spinal cage 10 has been reinforced with bioabsorbable polymer fibers 3 and ceramic fibers 4 embedded in a polymer matrix 2. The cage has been made by (a) winding a prepreg of matrix polymer, which is reinforced with continuous polymer and ceramic fibers, around a rectangular mold, (b) by melting the matrix polymer during the winding, and (c) by cooling the composite. Thereafter, the mold is -removed from the inside- of the rectangular composite tube and the tube has been cut to shorter cage samples.

FIG. 5 shows a perspective view of a cylindrical bar 11 of the invention, comprising a polymer matrix 2 and spirally wound polymer fibers 3 and ceramic fibers 4 embedded therein.

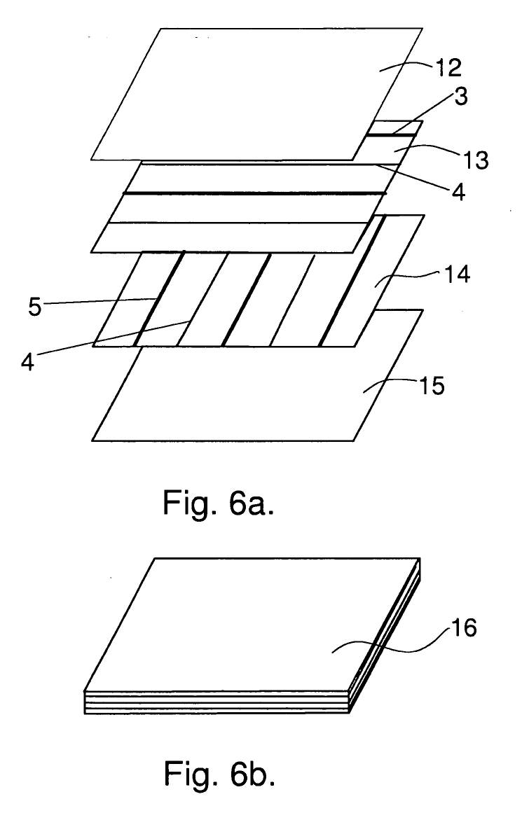

FIG. 6 shows a perspective view of a stack of 4 layers: an upper film 12 made of a matrix polymer, a polymeric prepreg 13 including polymer fibers 3 and ceramic fibers 4, a second polymeric prepreg 14 with polymeric fibers 3 and ceramic fibers 4 and a lower film 15. The films and prepregs can be compressed to a composite plate of the invention by using heat and pressure so that the upper and lower films 12 and 15 as well as the matrix of prepregs 13 and 14 melt at least partially and bind the polymeric and ceramic fiber 3 and 4 together to form a polymer matrix plate 16 (of FIG. 6b) reinforced with both polymer fibers and ceramic fibers.

Instead of matrix films it is also possible to use a matrix as fiber fabrics and to melt the fiber matrix to bind polymer and ceramic reinforcing fibers together.

Naturally, the composite materials of the invention can be fabricated by many other methods known in the polymer technology and/or in the composite technology as well as in the textile technology. For example, one advantageous method is injection molding, in which the polymer fiber+ a ceramic fiber insert is located inside the mold and a polymer melt is injected into the mold to fill the pores inside the fiber insert and the possible open space around the insert. Thereafter, the mold is cooled down so that the polymer melt (matrix) becomes solid and the composite sample can be removed from the mold.

The composite samples, such as membranes, meshes, foils, plates, rods or tubes, can be applied as implants in tissue fixation, regeneration or tissue generation.

The composite samples can be processed further mechanically and/or thermally into the form of more sophisticated implants to obtain e.g. screws, plates, nails, tacks, suture anchors, bolts, clamps, wedges, cages, etc. to be applied in different disciplines of surgery for tissue management, such as tissue fixation, or to help or guide tissue regeneration and/or generation.

### Examples

The following non-limiting examples give detailed information about the present invention.

### Example 1

Matrix: Poly-L/DL-lactide 70/30 (PLA₇₀), raw material from Boehringer Ingelheim, Germany (RESOMER®LR 708, Lot No. 290358, initial Mw ca. 370 000 Da (I.V.5.9-6.2 dl/g; when processed into form of flat strips MW ca. 215 000 Da)

Polymer fiber-reinforcement: Poly-L/D-lactide 96/4 raw material from Purac Biochem, the Netherlands (PURASORB® PLD, Lot No. 0209000939, initial I.V.5.48 dl/g; when processed into form of fibers Mw ca. 150 000 Da). The fibers with final diameter of ca. 85-95 μm were made by melt spinning with a single screw extruder.

Glass fiber reinforcement: Bioactive Glass 1-98 (53.0% SiO₂SiO₂SiO₂, 6.0% Na₂O, 22.0% CaO, 2.0% P₂O₅, 11.0% K₂O, 5.0% MgO, 1.0%, B₂O₃),. Bioactive glass fibers with the diameter of ca. 20-35 μm were manufactured at Tampere University of Technology (Institute of Biomaterials) by glass melt spinning.

Polymer reinforcement used to bind BaG-fibers: PLGA 50/50, raw material from Boehringer Ingelheim, Resomer® RG 503, Lot No. 10044449, I.V. 0,41 dl/g.

Test specimens, sized about 50×10×1.5 mm were manufactured by means of compression molding from preprocessed PLA₇₀ flat strips (44-53 wt-%), bioactive glass 1-98 (BaG) fibers (37-48 wt-%) and PLA₉₆ fibers (8-10 wt-%). PLA₇₀ flat strips were manufactured by extrusion and they acted as a matrix material. BaG (1-98) fibers were manufactured into a form of prepreg material during glass fiber processing binding them together with PLGA 50/50 (dissolved in acetone). The thickness of the prepregs was about 0.2 to 0.3 mm. The prepreg material was used as a reinforcement including the ceramic reinforcement component having unidirectional fiber alignment. 4-filament PLA₉₆ bundles were manufactured by means of fiber spinning, and they were further processed into the form of circular braids. These circular shaped braids were used as a continuous polymer fiber reinforcement covering PLA₇₀ flat strips. This means that the longest fibers covered the whole length of the final product. Finally all of these preforms were put into a mold (size 10 mm×50 mm) in a specific order:

(BaG/(PLA₉₆/PLA₇₀/PLA₉₆)/BaG/(PLA₉₆/PLA₇₀/PLA₉₆)/BaG)

After that the mold was heated to the desired temperature (139° C. to 141° C.) using a holding pressure of 5 MPa. When the desired temperature was achieved (typically after 3-5 min), the pressure was raised to the final value of 10 MPa and the mold was kept under heat and pressure for a prespecified time (1 min 30 s). After that the mold was cooled by using water cooling system.

Three point bending strength and modulus was measured for the test specimens using Instron 4411 Materials testing machine (Instron Ltd., High Wycombe, England). Pure PLA₇₀ without any reinforcing components was used as a reference material to the manufactured composites. The reference materials were extruded PLA₇₀ flat strips having the dimensions of about 50×8.3×1.5 mm.

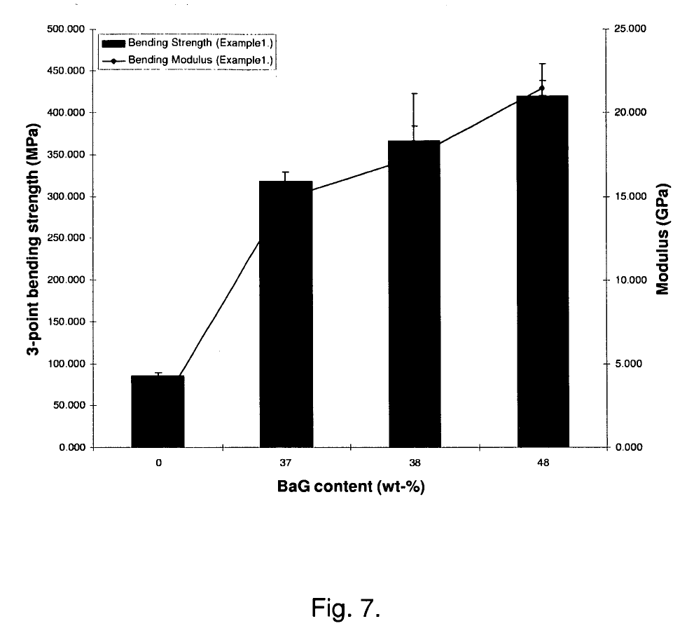

The maximum bending strength (yield) for the manufactured composites was 318.4 to 420.0 MPa and modulus 14.9 to 21.5 Gpa, depending on the BaG-fiber content. In comparison, the bending strength (yield) and modulus for pure PLA₇₀ were only 49.4 MPa and 2.2 GPa, respectively. Typical mechanical properties in 3-point bending test for the specimens of Example 1. are shown in Table 1. and in FIG. 1. The test specimens expressed fractures shortly after the maximum load, but the tough polymer reinforcement fibers prevented the fragmentation of the samples. Six parallel samples of pure PLA₇₀ and two parallel samples of other compositions were studied. FIG. 7 shows 3-point bending properties for manufactured composites. Error bars in FIG. 7 show standard deviations of the measurements.

TABLE 1

| | PLA₇₀ | PLA₉₆ | Load at | Stress at | | Strain at |
|---|---|---|---|---|---|---|
| BaG fiber | matrix | fiber | Yield | Yield (Max | Bending | Yield |
| content | content | content | (Max load) | Load) | Modulus | (MaxLoad) |
| wt-% | wt-% | wt-% | (N) | (MPa) | (GPa) | (mm/mm) |
| — | 100 | — | 49.4 ± 2.9 | 85.1 ± 4.2 | 2.2 ± 0.2 | 0.062 ± 0.002 |
| 37 | 53 | 10 | 184.2 ± 4.2 | 318.4 ± 10.6 | 14.9 ± 0.4 | 0.023 ± 0.001 |
| 38 | 52 | 10 | 220.5 ± 31.0 | 366.5 ± 56.7 | 17.4 ± 1.9 | 0.023 ± 0.001 |
| 48 | 44 | 8 | 287.7 ± 18.1 | 420.0 ± 39.1 | 21.5 ± 0.4 | 0.022 ± 0.003 |

### Example 2

Matrix: Poly-L/DL-lactide 70/30 (PLA₇₀), the same raw material from Boehringer Ingelheim, Germany, as in Example 1.

Polymer fiber-reinforcement: This was made of the same Poly-L/D-lactide 96/4 (from Purac Biochem, the Netherlands) as in Example 1.

Glass fiber reinforcement: Bioactive Glass 1-989898 fibers (diameter about 20-35 μm) were manufactured at Tampere University of Technology (Institute of Biomaterials) as in Example 1.

Polymer reinforcement used to bind BaG-fibers: PLGA 50/50, the same raw material from Boehringer Ingelheim as in Example 1.

Test specimens having dimensions of about 50×10×1.5 mm were manufactured in same fashion as in Example 1. from preprocessed PLA₇₀ flat strips (48 wt-%), bioactive glass 1-98 (BaG) fibers (42 wt-%) and PLA₉₆ fibers (10 wt-%). The only significant difference here was that the PLA96 fibers were discontinuous. Circular shaped braids were cut from one side so that their final shape was a flat braid or sheet composed of discontinuous 10-15 mm long fibers.

The strength of the polymer composites reinforced with discontinuous fibers can reach the strength of continuous fiber composites when the fiber length is approximately 10*I_{c} (where I_{c}=critical fiber length) and is 90% of the strength of 4*I_{c} (D. Hull). The critical length I_{c} of the fibers, which is defined as the minimum length of fiber required for stress to build up to the fracture strength (σ_{f}^{*}) of the fiber, is given by I_{c}=rσ_{f}*/τ

where r=radius of the fiber, and τ=the shear stress parallel to the fiber resisting pull-out (D. Hull).

The order of preforms which were laid into the mold (of Example 1):

(BaG/PLA₉₆/PLA₇₀/BaG/PLA₇₀/PLA₉₆/BaG)

The compression molding cycle for manufacturing the laminated specimens was identical to that of Example 1.The typical mechanical properties of samples for Example 2. (6 parallel samples) are shown in Table 2. The test specimens expressed fractures shortly after the maximum load but did not fragment. This behaviour was analogous with that of the samples in Example 1, while the reinforcing PLA96 fibers kept the fractured parts in position and prevented fragmentation.

TABLE 2

| | PLA₇₀ | PLA₉₆ | Load at | Stress at | | Strain at |
|---|---|---|---|---|---|---|
| BaG fiber | matrix | fiber | Yield | Yield (Max | | Yield |
| content | content | content | (Max load) | Load) | Modulus | (MaxLoad) |
| wt-% | wt-% | wt-% | (N) | (MPa) | (GPa) | (mm/mm) |
| 42 | 48 | 10 | 216.6 ± 21.8 | 378.2 ± 41.5 | 16.2 ± 1.4 | 0.025 ± 0.003 |

Reference: Hull D., An Introduction to Composite Materials, Cambridge University Press, Cambridge, UK, 1981, pp. 199-219.

### Example 3

Matrix: Poly-L/DL-lactide 70/30 (PLA₇₀), the same raw material from Boehringer Ingelheim, Germany, as above.

Polymer fiber-reinforcement: Poly-L/D-lactide 96/4, raw material from Purac Biochem, the Netherlands. Fibers were made as above.

Glass fiber reinforcement: Bioactive Glass 1-98 fibers (diameter about 20-35 μm) were manufactured at Tampere University of Technology (Institute of Biomaterials) as above.

Polymer reinforcement used to bind BaG-fibers: PLGA 50/50, the same raw material from Boehringer Ingelheim as in Example 1.

Test specimens having dimensions of about 50×10×2.6 mm were manufactured in the same fashion as in Example 1 from preprocessed PI-A₇₀ flat strips (52 wt-%), bioactive glass 1-98 (BaG) fibers (43 wt-%) and PLA₉₆ fibers (5 wt-%). The BaG prepreg material was here about 2-3 times thicker (thickness about 0.65 mm) than in Example 1. and this thicker prepreg material was used only on the top and bottom surfaces of the test specimens, while the BaG layer in the middle of the laminate composite was the same prepreg material as used in Example 1. The polymer fiber reinforcement here was continuous and it was introduced into the composite structure by covering PLA₇₀ flat strips by PLA₉₆ braids as in Example 1.

The order of preforms on a compression mold was:

(thick-BaG/(PLA₉₆/PLA₇₀/PLA₉₆)/thin-BaG/(PLA₉₆/PLA₇₀/PLA₉₆)/thick-BaG)

The compression molding cycle was the same as in Example 1 and in Example 2.

Table 3. shows the typical mechanical properties of samples of this Experiment (2 parallel samples).

The test specimens expressed fractures shortly after the maximum load, but in the same way as in Example 1. and Example 2., the reinforcing PLA₉₆ fibers kept the fractured parts in position and prevented fragmentation. An interesting finding was that although the BaG-fiber content was here smaller than that in the strongest test specimens of Example 1, the bending modulus was much higher. This indicates that the structure of the composite strongly affects its mechanical properties.

TABLE 3

| | PLA₇₀ | PLA₉₆ | Load at | Stress at | | Strain at |
|---|---|---|---|---|---|---|
| BaG fiber | matrix | fiber | Yield | Yield (Max | | Yield |
| content | content | content | (Max load) | Load) | Modulus | (MaxLoad) |
| wt-% | wt-% | wt-% | (N) | (MPa) | (GPa) | (mm/mm) |
| 43 | 52 | 5 | 339.8 ± 25.8 | 306.0 ± 18.4 | 27.2 ± 1.0 | 0.013 ± 0.001 |

### Example 4

Matrix: Poly-L/DL-lactide 70/30 (PLA₇₀), raw material from Boehringer Ingelheim (Germany), RESOMER® LR 708, Lot No. 290358, initial Mw about 370 000 Da (I.V. 5.9-6.2), (when processed into the form of flat strips MW about 215 000 Da). Matrix was melt processed into the form of thin (about 0.5 mm flat strips).

Polymer fiber-reinforcement: Poly-L/D-lactide 96/4, raw material from Purac Biochem (the Netherlands), PURASORB® PLD, Lot No. 0209000939, intial I.V. 5.48 dl/g (when processed into the form of fibers Mw about 150 000 Da, The fibers with final diameter of about 0.085-0.095 mm were made by melt spinning with a single screw extruder. PLA96 fibres were in the form of circular braids composed of 16 separate fibre bundles (8-filaments on each.)

Glass fiber reinforcement: Bioactive Glass 13-93 (53.0% SiO₂, 6.0% Na₂O, 20.0% CaO, 4.0% P₂O₅, 12.0% K₂O, 5.0% MgO) fibers with the diameter of ca. 20-35 μm were manufactured at Tampere University of Technology (Institute of Biomaterials) by glass melt spinning. Bioactive Glass fibres were in the form of sheets, in which the fibres were bound together using a solution of PLA70 (RESOMER® LR 708, Lot No. 290358) and acetone.

Test specimens, sized about 50×10×1.5 mm were manufactured by means of compression molding from preprocessed PLA₇₀ flat strips (about 40 wt-%), bioactive glass 13-93 (BaG) fibers (about 40 wt-%) and PLA₉₆ fibers (about 20 wt-%). The manufacturing methods of the raw materials and the compression molding cycle of composites were similar as in the previous examples (1-3).

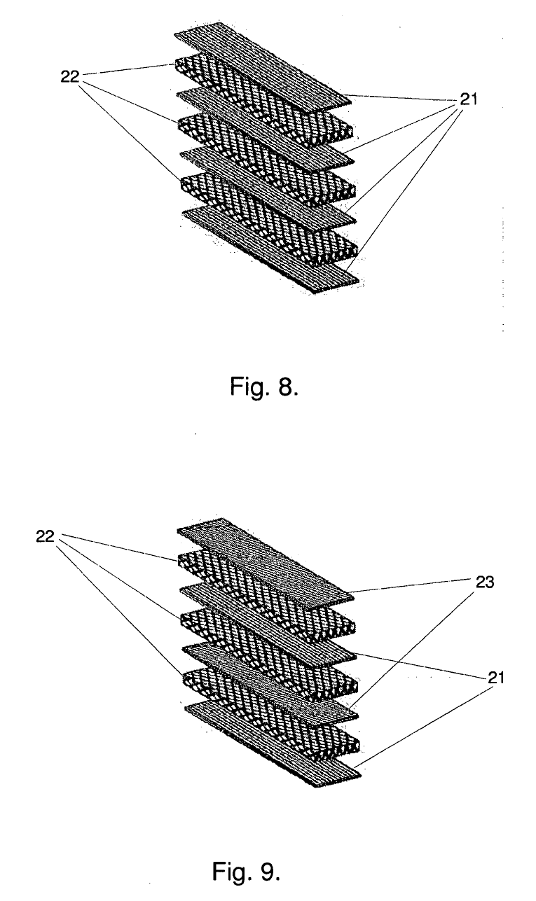

Three different compositions of BaG and PLA96 fibre reinforced composites were manufactured (Structures 1, 2 and 3, see FIGS. 8-10), and a plate composed of pure PLA70 (Structure 4, not shown) was used as a reference material.

Structure 1, which is shown in FIG. 8, comprises bioactive glass fibre sheets 21 aligned parallel to the longitudinal axis and strips 22. The strips 22 comprise a flat strip of PLA70 and PLA96 fibers which cover the flat strip. The PLA96 may form for example a braiding which is pulled over the flat strip. The PLA96 fibers form an angle with the longitudinal direction of the strip 22. The angle may be approximately 45°. Structure 2, shown in FIG. 9, also comprises strips 22 and bioactive glass fiber sheets 21 but half of the bioactive glass fiber sheets (sheets 23) are traverse to the longitudinal axis. Structure 3, shown in FIG. 10, comprises strips 21 and bioactive glass fiber sheets 23 aligned traverse to the longitudinal axis.

The aim of this example was to analyze the effect of the direction of BaG fibre reinforcement in impact resistance of composite structures. The test method used was IZOD impact strength measurement outlined in standard ISO 180. The alignment of PLA96 fibre reinforcements was similar in every composite structure analyzed (PLA70 matrix flat strips were covered by PLA96 fibre reinforcement). The number of parallel test specimens was 4 for all structures analyzed.

The measurement of impact strength was made for notched test specimens and determined using the IZOD method according to international standard ISO 180 (ISO 180:2000. Plastics -Determination of Izod impact strength. International Organization of Standardization 2000. p 1-10).

The thickness was, however, smaller than that mentioned in the standard (4 mm in ISO 180 and about 1.5 mm on this example). The measurements were made using Ceast Resil 5.5 testing machine (Pianezza-Torino, Italy). The pendulum struck the notched side of the specimens. The striking edge of the pendulum was 22 mm above the top plane of the support. The dimensions of the test specimens were 1.5×10×50 mm, the notch was 2 mm deep and the apex angle was 45°. The impact strength (J_{impact}) was expressed in kilojoules per square metre (kJ m⁻²) and calculated according to equation (1).

$${J}_{impact} = \frac{{E}_{measured} - {E}_{hammer}}{bh} \qquad ( 1 )$$

Where E_{measured} is the measured energy of impact, Eₕₐₘₘₑᵣ is the energy of the hammer without specimen, b is the sample thickness and h is the sample width. The used testing machine calculated the value for E_{measured}-Eₕₐₘₘₑᵣ automatically.

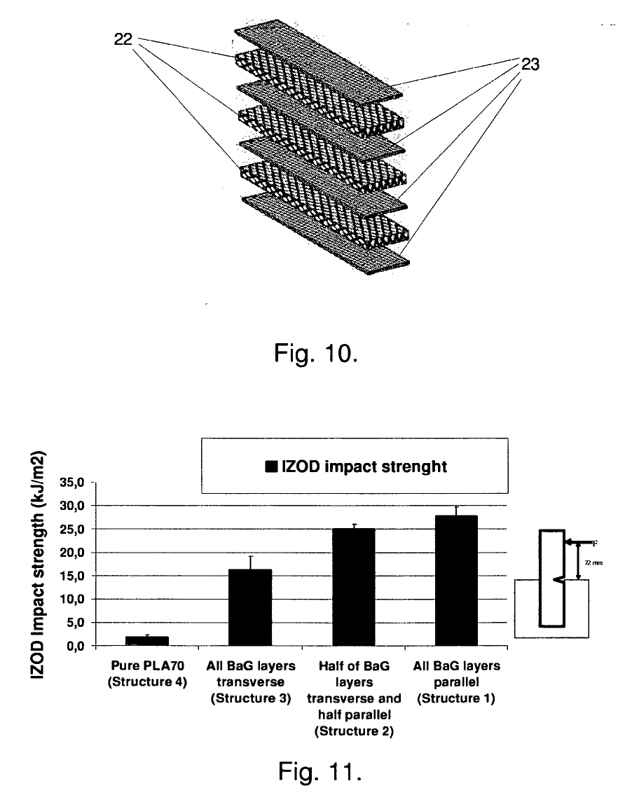

The results of impact strength measurements are presented in FIG. 11 and in Table 4. In FIG. 11 one can also see (on the right hand side) the principle of the test.

TABLE 4
Results of IZOD impact strength measurements and percentual
comparison to pure PLA70 and the effect of BaG fibre alignment.

| | | Percentual | Percentual | Percentual |
|---|---|---|---|---|
| | | increase in | increase in | increase in |
| | | Impact | Impact | Impact |
| | | resistance, | resistance, | resistance, |
| | Impact | compared to | compared to | compared to |
| | Strength | pure PLA70 | Structure 3 | Structure 2 |
| Structure | (kJ/m2) | (%) | (%) | (%) |
| Structure 4 | 1.8 ± 0.4 | | | |
| (pure | | | | |
| PLA70) | | | | |
| Structure 3 | 16.2 ± 3.0 | 786 | | |
| (All BaG | | | | |
| layers | | | | |
| transverse) | | | | |
| Structure 2 | 25.0 ± 1.1 | 1267 | 54 | |
| (Half of BaG | | | | |
| transverse | | | | |
| and half | | | | |
| parallel) | | | | |
| Structure 1 | 27.7 ± 2.1 | 1418 | 71 | 11 |
| (All BaG | | | | |
| layers | | | | |
| parallel) | | | | |

It can be seen in FIG. 11 and in Table 4 that double fibre reinforcing (BaG and PLA96 fibres) increased the impact strength of composites substantially. The reinforcing effect was 786 to 1418% depending on BaG fibre alignment when compared to test specimens composed of pure PLA70. The effect of BaG fibre alignment can also be seen clearly, as the impact strength was dependent on the alignment of BaG fibre-sheets; the impact resistance of Structure 1 was 54% higher than that of Structure 2 and 71% higher than that of Structure 3. In other words, the highest values were measured for composites having a BaG fibre alignment parallel to the longitudinal axis of composites, and the lowest values for double fibre reinforced composites were measured for structures in which all sheets of BaG fibres were aligned transverse to the longitudinal axis. The difference between Structure 1 and Structure 2 was 11%.

Besides the impact strength the fiber orientation has also another consequence. If all reinforcing fibers are parallel to each other and a fracture occurs in an implant, it will easily break apart. If there are fibers extending in different directions, the fracture cannot advance and the implant holds together. This is important because it has been reported that fragile ceramic implants have been broken in the system and have incurred the tetraplegia.

1. A bioabsorbable and bioactive composite material for surgical musculoskeletal applications comprising a bioabsorbable polymeric matrix material which is reinforced with bioabsorbable polymeric fibers and bioabsorbable ceramic fibers, characterized in that the surgical bioabsorbable polymeric matrix material is reinforced with the bioabsorbable polymeric fibers and the bioabsorbable ceramic fibers from which at least a portion is longer than 150 μm.

2. The composite material according to claim 1, characterized in that the amount of the bioabsorbable polymeric fibers, which are longer than 150 μm, is between 5 wt-% and 90 wt-% from the total weight of the composite.

3. The composite material according to claim 1 or 2, characterized in that the amount of the bioabsorbable ceramic fibers, which are longer than 150 μm, is between 10 wt-% and 90 wt-% from the total weight of the composite.

4. The composite material according to any preceding claim, characterized in that the polymeric matrix material is a homopolymer, a copolymer, a terpolymer, a polymer blend or a polymer alloy.

5. The composite material according to any preceding claim, characterized in that the bioabsorbable polymeric fibers are made of a homopolymer, or a copolymer, or a terpolymer, or a polymer blend or alloy.

6. The composite material according to any preceding claim, characterized in that the bioabsorbable ceramic reinforcing fibers are made of calcium phosphate and/or of a bioactive glass. 30

7. The composite material according to any preceding, characterized in that the bioabsorbable polymeric fibers and/or the ceramic reinforcing fibers are longer than 2 millimeters, preferably longer than 30 millimetres.

8. The composite material according to any preceding claim, characterized in that the bioabsorbable polymeric fibers and/or ceramic fibers are continuous in the composite structure.

9. The composite material according to any preceding claim, characterized in that the diameter of the bioabsorbable polymeric reinforcing fibers is between 4 μm and 800 μm, preferably between 20 μm and 500 μm.

10. The composite material according to any preceding claim, characterized in that the diameter of the bioceramic fibers is between 2 μm and 500 μm, preferably between 20 μm and 200 μm.

11. The composite material according to any preceding claim, characterized in that its bending modulus, as measured at RT with a three point bending test, is at least 15 GPa.

12. The composite material according to any preceding claim, characterized in that the composite material contains at least one pharmaceutically active agent in the bioabsorbable polymer matrix and/or in the bioabsorbable polymeric fibers.

13. The composite material according to any preceding claim, characterized in that the composite comprises in the polymer matrix a bioabsorbable ceramic powder or bioabsorbable ceramic fibers, which are shorter than 150 μm.

14. The composite material according to claim 1, characterized in that
the surgical bioabsorbable polymeric matrix material comprises layers which are laminated together and reinforced with the bioabsorbable polymeric fibers and/or the bioabsorbable ceramic fibers, the bioabsorbable polymeric fibers and the bioabsorbable ceramic fibers being continuous in the layer, and
the superimposed layers comprise layers which differ from each other in their fiber orientation.

15. A method for manufacturing a bioabsorbable and bioactive composite material according to claim 1, comprising
selecting at least one bioabsorbable polymer for the polymer matrix;
selecting bioabsorbable polymer fibers having a length which is longer than 150 μm for use as the polymeric reinforcing fibers;
selecting bioceramic fibers having a length which is longer than 150 μm for use as the ceramic reinforcing fibers;
aligning or mixing said first polymer and said second bioabsorbable polymer fibers and said bioceramic fibers together to form a mixture;
placing said mixture into a desired mold or die; and
subjecting the mixture to heat and/or pressure and/or mechanical force to yield the bioabsorbable and bioactive composite material.
